(19) United States
(12) Patent Application Publication
(10) Pub. No.: US 2017/0165434 A1
(43) Pub. Date: Jun. 15, 2017

(54) MULTI-NEEDLE NEEDLE ASSEMBLY

(71) Applicant: Xiamen Brana Design Co., Ltd., Xiamen City (CN)

(72) Inventor: Zhongtang Wang, Xiamen City (CN)

(21) Appl. No.: 15/372,419

(22) Filed: Dec. 8, 2016

(30) Foreign Application Priority Data
Dec. 10, 2015 (CN) 201510910198.1
Dec. 10, 2015 (CN) 201510910212.8

(51) Int. Cl. A61M 5/32; A61M 5/46; A61M 5/34

(52) U.S. Cl. CPC A61M 5/3295; A61M 5/34; A61M 5/3291; A61M 5/46

(57) ABSTRACT

A multi-needle needle assembly includes a multi-needle body, a box, and a needle length adjustment mechanism. The multi-needle body is disposed in the box. The multi-needle body includes a plurality of needles and a connecting opening for connecting an automatic auxiliary device, such that the effect of full-thickness skin injection can be achieved to save injection time.

### FIELD OF THE INVENTION

The present invention relates to a medical needle, and more particularly to a multi-needle needle assembly for facial or body surface injection in the plastic surgery field, such as hyaluronic acid, collagen, botulinum toxin, PRP, autologous cells. It also can be used for injection of body surface scar medicine and injection of vaccine.

### BACKGROUND OF THE INVENTION

The cumulative effect of UV damage, skin collagen loss, subcutaneous fat atrophy, skin thinning, water retention weakened, weakened skin elasticity, dark spots, and wrinkles increase with age.

These days, the requirements for skin beauty injection include non-surgical treatment, non-invasive, painless, no trace; beauty preparation from deep to shallow, full-thickness three-dimensional skin injection; multiple effects, filling, water replenishment, skin whitening, tender skin, shrinkage of pores, wrinkle removal, and so on; in a simple and quick way.

The current solutions include: oral collagen, hyaluronic acid; local smudge therapy; transdermal patch therapy; needling; laser drilling; local manual injection; and automated instrument assist injection. Clinical practice shows that oral collagen, hyaluronic acid and other cosmetic preparations did not effect. The local smudge therapy only reaches the cuticle of the skin, and the effect only maintains about one day. As to the transdermal patch therapy, it only reaches the superficial epidermis, and the preparation must be fat-soluble. Most of the cosmetic preparations are water-soluble. The damage of the needling is large. The heat damage of the laser is the residual coloring. The local manual injection is reliable, but the depth of injection is not easy to control. The single-needle injection makes the operator tired easily. As to the multi-needle automatic injection by means of negative pressure, the depth of injection can be controlled, with high efficiency, and the operator won't be fatigued.

A conventional multi-needle needle is able to adjust the length of the needle within a certain range, achieving different depth of skin injection. Full-thickness three-dimensional skin injection has better cosmetic results. The operator adjusts the length of the needle for a skin deep injection, re-adjusts the length of the needle for a skin dermis middle injection, and then re-adjusts the length of the needle, for a skin dermis shallow injection. Despite the support of the equipment, it takes three times as long as the single-level injection. This is quite time-consuming and laborious.

In addition, the conventional multi-needle needle must be connected with the equipment for use, and it cannot be connected with a syringe for manual operation. Because of a variety of factors, such as inconvenience of carrying the equipment, malfunction of the equipment, daily use habits and high price of the equipment, the advance technique cannot be widely used.

Accordingly, the inventor of the present invention has devoted himself based on his many years of practical experiences to solve these problems.

### SUMMARY OF THE INVENTION

The primary object of the present invention is to provide a multi-needle needle assembly with high efficiency.

In order to achieve the foregoing object, the multi-needle needle assembly of the present invention comprises a multi-needle body, a box, and a needle length adjustment mechanism. The multi-needle body is disposed in the box. The multi-needle body comprises a connecting opening for connecting a device or a syringe, a fluid guide chamber, a needle seat, and a plurality of needles. An inlet end of the fluid guide chamber is communicated with the connecting opening. An outlet end of the fluid guide chamber is connected with the needle seat and communicated with a passage inside the needle seat. Inlet ends of the plurality of needles are spaced apart from each other and perpendicularly fixed to the needle seat. The inlet ends of the plurality of needles are communicated with the passage of the needle seat. The needle length adjustment mechanism comprises a knob disposed on the needle seat of the multi-needle body and a slot disposed on a side wall of a leading-in chamber of the box. The knob is inserted in the slot. A wall surface of the slot is provided with a scale mark.

Preferably, an end of the box is provided with a negative pressure chamber. A side wall of the box is formed with a negative pressure inlet communicating with the negative pressure chamber. Another end of the box is provided with the leading-in chamber. The multi-needle body is movably connected to the leading-in chamber. A limit plate is provided between the negative pressure chamber and the leading-in chamber. The limit plate is formed with a plurality of needle holes. The needle seat of the multi-needle body is movably disposed in the leading-in chamber of the box. The plurality of needles of the multi-needle body are spaced and inserted through the plurality of needle holes of the limit plate to enter the negative pressure chamber of the box.

Preferably, the plurality of needles have a same length, and a side wall of each needle is formed with a plurality of side apertures spaced at a determined distance apart from each other.

Preferably, the side apertures of each needle are disposed close to a needle point thereof, and a distance between every adjacent two of the side apertures is 0.2-0.5 mm.

Preferably, the plurality of needle holes of the limit plate are arranged in an array, and a distance between every adjacent two of the needle holes is 5 mm.

Preferably, the plurality of needles have different lengths and are divided into three types according to their respective lengths, and the three types of needles are staggered and arranged in an array.

Preferably, the three types of needles have a difference of 0.4-0.6 mm in length.

Accordingly, since the multi-needle body of the present invention comprises the plurality of needles. The connecting opening of the multi-needle body can be connected with an automatic auxiliary device, such that full-thickness skin injection effect can be achieved after the connection of the device to save operation time. In addition, the present invention further includes the needle length adjustment mechanism, which makes the box very simple without the negative pressure chamber, the negative pressure inlet and a fixing device, and has the function of needle length adjustment and can be manually operated by connecting a conventional syringe. A single injection can achieve the effect of full-thickness skin injection. The multi-needle needle assembly of the present invention can improve the working efficiency for the operator, reduce labor intensity, and achieve a negative pressure injection and a manual injection.

### BRIEF DESCRIPTION OF THE DRAWINGS

FIG. 1 is a perspective view of the present invention;

FIG. 2 is a perspective sectional view according to a first embodiment of the present invention;

FIG. 3 is a front view according to the first embodiment of the present invention;

FIG. 4 is a side view according to the first embodiment of the present invention;

FIG. 5 is a front view showing the multi-needle body according to the first embodiment of the present invention;

FIG. 6 is a front view showing the needle according to the first embodiment of the present invention;

FIG. 7 is a perspective sectional view according to a second embodiment of the present invention;

FIG. 8 is a front view according to the second embodiment of the present invention;

FIG. 9 is a side view according to the second embodiment of the present invention; and

FIG. 10 is a front view showing the multi-needle body according to the second embodiment of the present invention.

### DETAILED DESCRIPTION OF THE PREFERRED EMBODIMENTS

Embodiments of the present invention will now be described, by way of example only, with reference to the accompanying drawings.

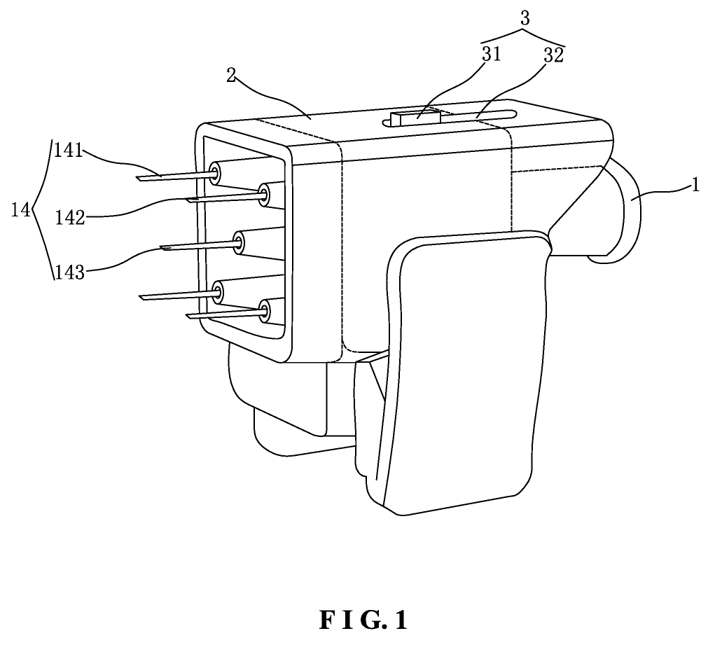

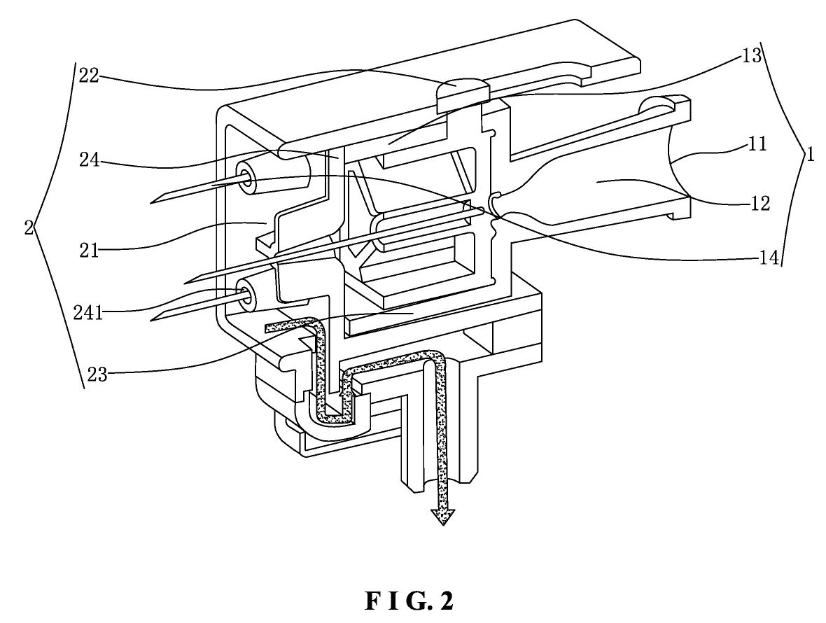

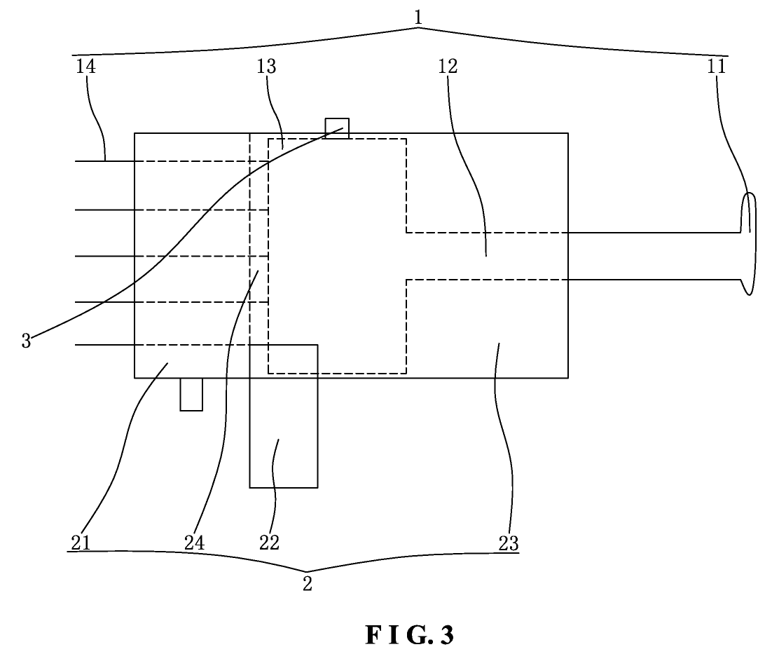

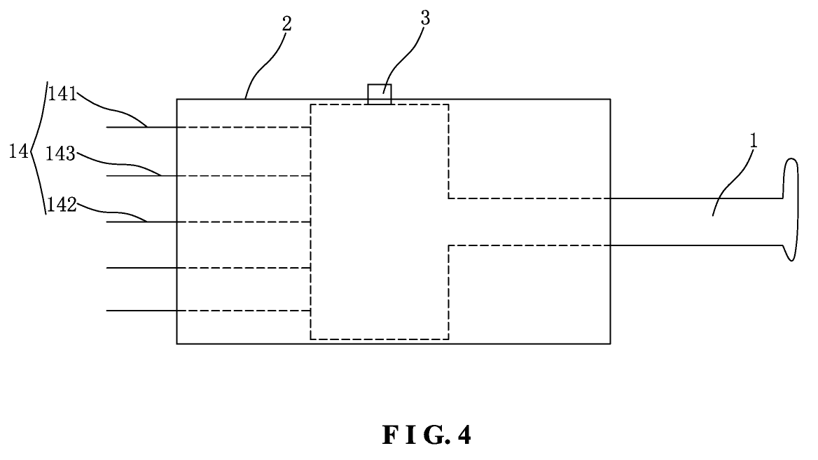

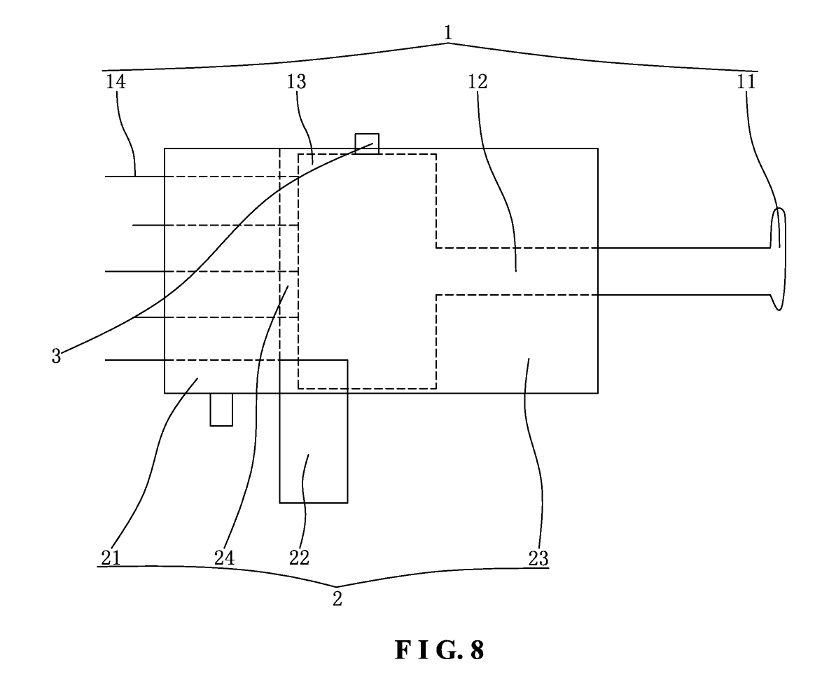

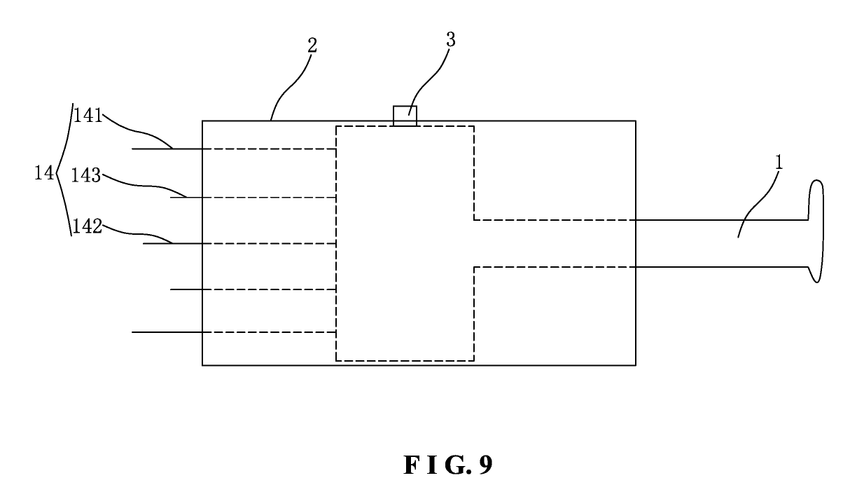

As shown in FIG. 1 through FIG. 10, the present invention discloses a multi-needle needle assembly. The multi-needle needle assembly comprises a multi-needle body 1, a box 2, and a needle length adjustment mechanism 3.

The multi-needle body 1 is disposed in the box 2.

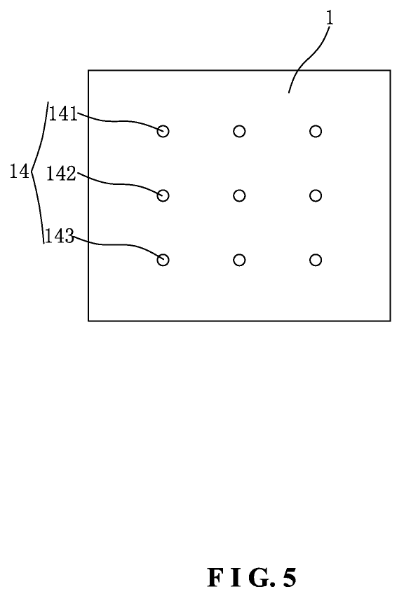

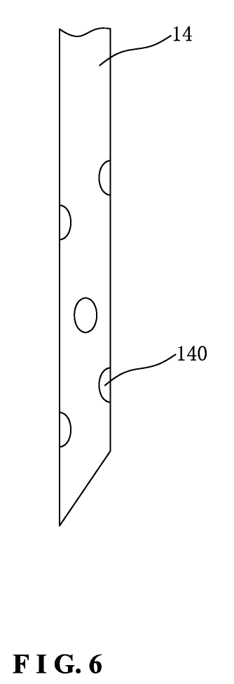

FIG. 1 to FIG. 6 shows a first embodiment of the present invention. The multi-needle body 1 comprises a connecting opening 11, a fluid guide chamber 12, a needle seat 13, and a plurality of needles 14. An inlet end of the fluid guide chamber 12 is the connecting opening 11 to connect with a device or a syringe. The connecting opening 11 may be a threaded connecting opening or a snap-on connecting opening. An outlet end of the fluid guide chamber 12 is connected with the needle seat 13 and communicated with a passage inside the needle seat 13. Inlet ends of the plurality of needles 14 are spaced apart from each other and perpendicularly fixed to the needle seat 13. The plurality of needles 14 are arranged in an array. The inlet ends of the plurality of needles 14 are communicated with the passage of the needle seat 13. Fluid enters from the connecting opening 11, passes the passage of the needle seat 13, and diverges into the plurality of needles 14. In this embodiment, in cooperation with FIG. 5, the number of the plurality of needles 14 is a multiple of three, and is divided into three groups. In this embodiment, there are nine needles 14. Each group has three needles 14. The needles 14 of the three groups are arranged in a staggered array. The needles 14 of each group have the same length. As shown in FIG. 6, the side wall of the needle 14 is formed with a plurality of side apertures 140.

An end of the box 2 is provided with a negative pressure chamber 21. A side wall of the box 2 is formed with a negative pressure inlet 22 communicating with the negative pressure chamber 21. Another end of the box 2 is provided with a leading-in chamber 23. The multi-needle body 1 is movably connected to the leading-in chamber 23. A limit plate 24 is provided between the negative pressure chamber 21 and the leading-in chamber 23. The limit plate 24 is formed with a plurality of needle holes 241. In this embodiment, the limit plate 24 has nine needle holes 241. The plurality of needle holes 241 are arranged in an array. The distance between every adjacent two of the needle holes 241 is 5 mm. The needle seat 13 of the multi-needle body 1 is movably disposed in the leading-in chamber 23 of the box 2. The plurality of needles 14 of the multi-needle body 1 are spaced and inserted through the plurality of needle holes 241 of the limit plate 24 to enter the negative pressure chamber 21 of the box 2.

The needle length adjustment mechanism 3 comprises a knob 31 disposed on the needle seat 13 of the multi-needle body 1 and a slot 32 disposed on the side wall of the leading-in chamber 23 of the box 2. The knob 31 is inserted in the slot 32. The wall surface of the slot 32 is provided with a scale mark. The knob 31 is a screw-type structure.

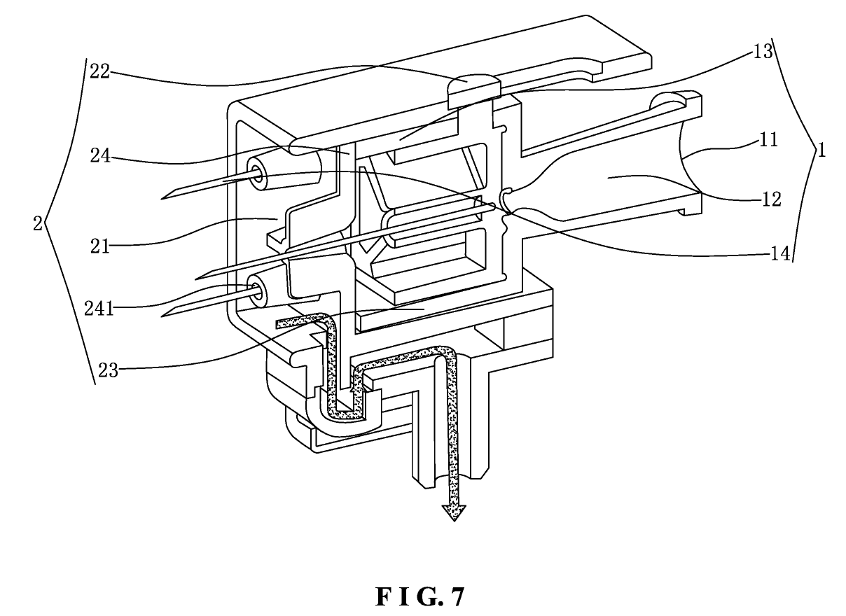

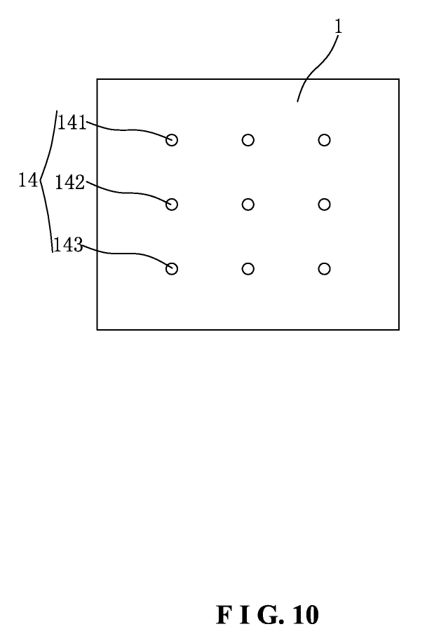

FIG. 7 to FIG. 10 in cooperation with FIG. 1 shows a second embodiment of the present invention. The multi-needle body 1 comprises a connecting opening 11, a fluid guide chamber 12, a needle seat 13, and a plurality of needles 14. An inlet end of the fluid guide chamber 12 is the connecting opening 11 to connect with a device or a syringe. The connecting opening 11 may be a threaded connecting opening or a snap-on connecting opening. An outlet end of the fluid guide chamber 12 is connected with the needle seat 13 and communicated with a passage inside the needle seat 13. Inlet ends of the plurality of needles are spaced apart from each other and perpendicularly fixed to the needle seat 13. The plurality of needles 14 are arranged in an array. The inlet ends of the plurality of needles 14 are communicated with the passage of the needle seat 13. Fluid enters from the connecting opening 11, passes the passage of the needle seat 13, and diverges into the plurality of needles 14. In this embodiment, in cooperation with FIG. 10, the plurality of needles 14 are divided into three types. Each type of the needle 14 has a different length, namely, a long needle 141, a middle needle 142 and a short needle 143. The difference in length of each needle 14 is determined by the application, with a difference of 0.4-0.6 mm. The three types of the needles 14 are arranged in a staggered array. The number of needles 14 is a multiple of three. In is embodiment, there are nine needles 14. The number of each type of the needles 14 is three.

An end of the box 2 is provided with a negative pressure chamber 21. A side wall of the box 2 is formed with a negative pressure inlet 22 communicating with the negative pressure chamber 21. Another end of the box 2 is provided with a leading-in chamber 23. The multi-needle body 1 is movably connected to the leading-in chamber 23. A limit plate 24 is provided between the negative pressure chamber 21 and the leading-in chamber 23. The limit plate 24 is formed with a plurality of needle holes 241. In this embodiment, the limit plate 24 has nine needle holes 241. The plurality of needle holes 241 are arranged in an array. The distance between every adjacent two of the needle holes 241 is 5 mm. The needle seat 13 of the multi-needle body 1 is movably disposed in the leading-in chamber 23 of the box 2. The plurality of needles 14 of the multi-needle body 1 are spaced and inserted through the plurality of needle holes 241 of the limit plate 24 to enter the negative pressure chamber 21 of the box 2.

The needle length adjustment mechanism 3 comprises a knob 31 disposed on the needle seat 13 of the multi-needle body 1 and a slot 32 disposed on the side wall of the leading-in chamber 23 of the box 2. The knob 31 is inserted in the slot 32. The wall surface of the slot 32 is provided with a scale mark. The knob 31 is a screw-type structure.

It is noted that the box of the present invention may be connected to a conventional syringe to perform a manual operation. There is no need to provide the negative pressure chamber, the negative pressure inlet, and a fixing device for connection of the multi-needle body.

The working principle of the present invention is described below.

The connecting end 11 of the multi-needle body 1 is externally connected with a device or a syringe. The syringe may be connected to the connecting opening by means of a snap-on way or threads. The plurality of needles 14 of the multi-needle body 1 are spaced and inserted through the plurality of needle holes 241 of the limit plate 24 to enter the negative pressure chamber 21 of the box 2. The negative pressure inlet 22 of the box 2, communicating with the negative pressure chamber 21, is connected with a negative pressure device. The negative pressure chamber 21 of the box 2 is attached to the patient's skin. The patient's skin under the action of negative pressure of the negative pressure chamber 21 will make the patient's skin lean against the plurality of needles 14 for injection. The fluid is injected from the connecting opening 11 through the passage of the needle seat 13 into the patent's skin by means of the plurality of needles 14.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A multi-needle needle assembly, comprising a multi-needle body, a box, and a needle length adjustment mechanism;
the multi-needle body being disposed in the box;
the multi-needle body comprising a connecting opening for connecting a device or a syringe, a fluid guide chamber, a needle seat, and a plurality of needles, an inlet end of the fluid guide chamber being communicated with the connecting opening, an outlet end of the fluid guide chamber being connected with the needle seat and communicated with a passage inside the needle seat, inlet ends of the plurality of needles being spaced apart from each other and perpendicularly fixed to the needle seat, the inlet ends of the plurality of needles being communicated with the passage of the needle seat;
the needle length adjustment mechanism comprising a knob disposed on the needle seat of the multi-needle body and a slot disposed on a side wall of a leading-in chamber of the box, the knob being inserted in the slot, a wall surface of the slot being provided with a scale mark.

2. The multi-needle needle assembly as claimed in claim 1, wherein an end of the box is provided with a negative pressure chamber, a side wall of the box is formed with a negative pressure inlet communicating with the negative pressure chamber, another end of the box is provided with the leading-in chamber, the multi-needle body is movably connected to the leading-in chamber, a limit plate is provided between the negative pressure chamber and the leading-in chamber, the limit plate is formed with a plurality of needle holes; the needle seat of the multi-needle body is movably disposed in the leading-in chamber of the box, and the plurality of needles of the multi-needle body are spaced and inserted through the plurality of needle holes of the limit plate to enter the negative pressure chamber of the box.

3. The multi-needle needle assembly as claimed in claim 1, wherein the plurality of needles have a same length, and a side wall of each needle is formed with a plurality of side apertures spaced at a determined distance apart from each other.

4. The multi-needle needle assembly as claimed in claim 3, wherein the side apertures of each needle are disposed close to a needle point thereof, and a distance between every adjacent two of the side apertures is 0.2-0.5 mm.

5. The multi-needle needle assembly as claimed in claim 1, wherein the plurality of needle holes of the limit plate are arranged in an array, and a distance between every adjacent two of the needle holes is 5 mm.

6. The multi-needle needle assembly as claimed in claim 1, wherein the plurality of needles have different lengths and are divided into three types according to their respective lengths, and the three types of needles are staggered and arranged in an array.

7. The multi-needle needle assembly as claimed in claim 6, wherein the three types of needles have a difference of 0.4-0.6 mm in length.
